(19) United States
(12) Patent Application Publication
(10) Pub. No.: US 2003/0017504 A1
(43) Pub. Date: Jan. 23, 2003

(54) Antibodies specific for poly(ethylene glycol)

(75) Inventors: Michael J. Roberts, Williamsburg, VA (US); Mary Elizabeth Green, Ardmore, AL (US)

Correspondence Address: ALSTON & BIRD LLP, BANK OF AMERICA PLAZA, 101 SOUTH TRYON STREET, SUITE 4000, CHARLOTTE, NC, 28280-4000, US

(73) Assignee: SHEARWATER CORPORATION

(21) Appl. No.: 10/153,024

(22) Filed: May 21, 2002

Related U.S. Application Data
(60) Provisional application No. 60/292,371, filed on May 21, 2001.

(51) Int. Cl. G01N 33/53; C07K 16/00

(52) U.S. Cl. 435/7.1; 530/388.1

(57) ABSTRACT

The present invention provides IgG monoclonal antibodies and IgG monoclonal antibody fragments that selectively bind to PEG, hybridoma cell lines and methods for producing these antibodies, and methods for using the PEG-selective monoclonal antibodies. The methods of the invention include methods for detecting molecules, viruses, cells, or organelles that contain at least one poly(ethylene glycol) group and methods for purifying for purifying molecules, viruses, cells, or organelles that contain at least one poly(ethylene glycol) group. Also provided are methods for using the anti-PEG monoclonal antibodies in vivo to modulate the level of a PEG-containing molecule.

### CROSS-REFERENCE TO RELATED APPLICATIONS

This application claims the benefit of Provisional Application Serial No. 60/292,371, filed May 21, 2001, which is incorporated by reference in its entirety.

### FIELD OF THE INVENTION

The invention relates to monoclonal antibodies and antibody fragments that bind selectively to poly(ethylene glycol).

### BACKGROUND OF THE INVENTION

Chemical attachment of the hydrophilic polymer poly(ethylene glycol) (“PEG”) to molecules and surfaces is of great utility in biotechnology. In its most common form PEG is a linear polymer terminated at each end with hydroxyl groups:

HO—CH₂CH₂O—(CH₂CH₂O)ₙ—CH₂CH₂—OH

Typically, n ranges from about 10 to about 2000.

PEG is commonly used as methoxy PEG-OH, or mPEG in brief, in which one terminus is the relatively inert methoxy group, while the other terminus is a hydroxyl group that is subject to ready chemical modification.

CH₃O—(CH₂CH₂O)ₙ—CH₂CH₂—OH mPEG

PEG is also commonly used in branched forms that can be prepared by addition of ethylene oxide to various polyols, such as glycerol, pentaerythritol and sorbitol. Branched PEGs can also be prepared by attaching two PEG “arms” to a central linking moiety having a single functional group capable of joining to other molecules.

PEG is a well known polymer having the properties of solubility in water and in many organic solvents, lack of toxicity, and lack of immunogenicity. One use of PEG is to covalently attach the polymer to insoluble molecules to make the resulting PEG-molecule “conjugate” soluble. For example, it has been shown that the water-insoluble drug paclitaxel, when coupled to PEG, becomes water-soluble (Greenwald et al. (1995) J. Org. Chem., 60:331-336).

In related work, U.S. Pat. No. 4,179,337 to Davis et al. discloses that proteins coupled to PEG have enhanced blood circulation lifetime because of reduced rate of kidney clearance and reduced immunogenicity. These and other applications are also described in Biomedical and Biotechnical Applications of Poly(ethylene glycol) Chemistry, J. M. Harris, Ed., Plenum, New York (1992), and Poly(ethylene glycol) Chemistry and Biological Applications, J. M. Harris and S. Zalipsky, Eds., ACS, Washington D.C. (1997).

Because of the advantages conferred by PEG modification, there is a need for reagents and methods useful for specifically binding, detecting, and purifying PEG-modified molecules.

### SUMMARY OF THE INVENTION

The present invention provides IgG monoclonal antibodies and IgG monoclonal antibody fragments that selectively bind to poly(ethylene glycol), hybridoma cell lines and methods for producing these antibodies, and methods for using the poly(ethylene glycol)-selective monoclonal antibodies.

The invention provides the amino acid sequence of a light chain variable region (SEQ ID NO:1) and heavy chain variable region (SEQ ID NO:2) of an IgG monclonal antibody that binds selectively to poly(ethylene glycol). These amino acid sequences, variants and fragments thereof are encompassed by the present invention. Nucleotide sequences encoding these amino acid sequences and methods for producing recombinant antibodies comprising these sequences are also encompassed.

In one embodiment, the anti-PEG IgG monoclonal antibodies or anti-PEG monoclonal antibody fragments comprise at least one of the amino acid sequences set forth in SEQ ID NO:1 or SEQ ID NO:2, or a variant of SEQ ID NO: 1 or SEQ ID NO:2.

Hybridoma cell lines producing IgG monoclonal antibodies that bind selectively to PEG are also encompassed by the present invention. In one embodiment, the hybrdioma cell line is the one deposited with the ATCC as Patent Deposit Number PTA-4112 on Mar. 1, 2002.

The invention provides methods for detecting targets that contain at least one PEG group. The target may be, for example, molecules, virus particles, cells, or organelles. Exemplary targets include peptides, proteins, enzymes, cytokines, hematopoietins, growth factors, hormones, antigens, antibodies, antibody fragments, receptors, protein fragments, drugs, dyes, nucleotides, oligonucleotides, saccharides, polysaccharides, natural polymers, synthetic polymers, lipids, and phospholipids. In one embodiment, the methods comprise the steps of contacting the target with an anti-PEG IgG monoclonal antibody under conditions such that the antibody can bind to the PEG group contained in the target, and detecting the resulting complex by an immunoassay.

The invention also provides methods for purifying targets that contain at least one PEG group. Examples of targets that may be purified include molecules, virus particles, cells, or organelles. In one embodiment, the methods comprise the steps of contacting the target with an anti-PEG IgG monoclonal antibody under conditions such that the antibody can bind to the PEG group contained in the target, and recovering the resulting complex.

In another embodiment of the invention, the antibodies are used ex vivo as affinity ligands in chromatography or in an enzyme-linked immunosorbent assay (ELISA) as well as many other diagnostic, analytical, or characterization techniques known in the art where antibodies or their fragments are used.

The invention also encompasses the use of such IgG monoclonal antibodies or antibody fragments thereof selective for poly(ethylene glycol) in therapeutic or diagnostic applications. In one embodiment, the anti-PEG antibodies are used in vivo for binding to and tracking or clearing poly(ethylene glycol) modified molecules. In one embodiment, the anti-PEG IgG monoclonal antibodies and antibody fragments are partially or fully humanized or fully human for human in vivo applications.

### BRIEF DESCRIPTION OF THE DRAWING

Having thus described the invention in general terms, reference will now be made to the accompanying figures, wherein:

FIG. 1 shows a Western blot analysis demonstrating that the mouse anti-PEG monoclonal antibody 157D 29G1 binds selectively to PEG. The gel was loaded in the following order: lane 1—prestained protein standard, lane 2—bovine serum albumin (BSA), lane 3—BSA-PEG, lane 4—Lys, lane 5—Lys-PEG, and lanes 6 to 10, PEG molecular weights of 2, 5, 10, 20 and 30 kDa, respectively.

### DETAILED DESCRIPTION OF THE INVENTION

I. Definitions

The following words and phrases have the definitions that are indicated and are used in the entire application.

The term “antibody” is used herein to describe proteins composed of at least one light chain (L) and at least one heavy chain (H) that exhibit binding specificity to a specific antigen. There are five classes of immunoglobulins: IgG, IgA, IgE, IgD, IgM, and several can be further divided into subclasses. Examples of the types of antibodies include full-length antibodies, antibody fragments, single chain antibodies, multispecific or multifunctional antibodies, diabodies, chimeric antibodies, humanized antibodies, and human antibodies.

The term “monoclonal antibody” is used to describe a preparation of antibody molecules produced by a hybridoma. In contrast with polyclonal antibody preparations that typically include different antibodies directed against different determinants (epitopes), each monoclonal antibody is directed against a single determinant on the antigen.

The term “hybridoma” is used to describe a clone of hybrid cells formed by fusion of normal lymphocytes with myeloma cells. The hybridoma retains the properties of the normal cell in order to produce antibodies but exhibits the immortal growth characteristic of myeloma cells.

The term “full-length antibody” is defined as an immunoglobulin molecule composed of two heavy chains (H) of at least about 50,000 Da MW and two light chains (L) of at least about 25,000 Da held together by multiple interchain disulfide bonds. Starting at the N-terminus, the light chain can be divided into two domains, a variable domain (V_{L}) comprising of about 110 amino acids and a constant domain (C_{L}) comprising of about the same number of amino acids. The heavy chain can be divided in a similar way, but the heavy chain consists of one variable domain (V_{H}) and three constant domains (C_{H}1, C_{H}2 and C_{H}3).

“Antibody fragments” are defined as portions of an intact full-length antibody comprising the antigen binding site and include Fv, Fv′, Fab, Fab′, F(ab′)₂, single chain Fv molecules (scFv), light chain variable regions without an associated heavy chain, or a heavy chain variable region without an associated light chain.

A “multispecific” or “multifunctional antibody” is a molecule that exhibits at least two different binding specificities to at least two different epitopes. The at least two different epitopes can be from the same antigen molecule or different antigen molecules.

A “chimeric antibody” is an antibody molecule that is composed of a human Fc region and a non-human (eg. rodent or primate) V_{L} and V_{H} regions. A “humanized antibody” is an antibody molecule that is composed of a human immunoglobulin in which residues from a complementary determining region (CDR) are replaced by residues from a CDR of a non-human species having the desired specificity, affinity and capacity.

A “purified antibody” as used herein refers to an antibody that is substantially free of cellular material or other contaminating proteins from the cell or other source from which the antibody is derived, or substantially free from chemical precursors or other chemicals when chemically synthesized. Amino acids are listed by either the three letter or single letter abbreviations: Glycine (Gly, G), Alanine (Ala, A), Valine (Val, V), Leucine (Leu, L), Isoleucine (Ile, I), Methionine (Met, M), Proline (Pro, P), Phenylalanine,(Phe, F), Tryptophan (Trp, W), Serine (Ser, S), Threonine (Thr, T), Asparagine (Asn, N), Glutamine (Gln, Q), Tyrosine, (Tyr, Y), Cysteine (Cys, C), Lysine (Lys, K), Arginine (Arg, R), Histidine (His, H), Aspartic Acid (Asp, D), and Glutamic acid (Glu, E).

Nucleic Acids are listed by the single letter abbreviations: Adenine (A), Cytosine (C), Guanine, (G), Thymine (T), and Uracil (U).

II. Immunization of Host Animals

The present invention provides monoclonal antibodies that selectively bind to poly(ethylene glycol) of various molecular weights were isolated from hybridoma cell lines formed by the fusion of antigen-primed spleen cells with myeloma cells. See, , for example, Example 3. The hybridoma technique used to prepare the antibodies of the invention was described originally by Kohler and Milstein, Eur. J. Immunol. 6, 511 (1976) and has been widely applied to produce hybridoma cell lines that secrete high levels of monoclonal antibodies against specific antigens. Additional techniques such as phage display are contemplated to produce IgG monoclonal antibodies or fragments thereof specific for poly(ethylene glycol).

Immunization of the host animal or cultured antibody-producing cells therefrom is generally in keeping with established and conventional protocols for antibody stimulation and production. The applicants have employed mice as the test model although it is contemplated that any mammalian subject, including human subjects or antibody producing cells therefrom, can be manipulated according to the processes of this invention to serve as the basis for production of mammalian, including human, hybridoma cell lines.

III. Hybridoma Cell Lines

After immunization, immune lymphoid cells are fused with myeloma cells to generate a hybrid cell line that can be cultivated and subcultivated indefinitely, to produce large quantities of monoclonal antibodies. For purposes of this invention, the immune lymphoid cells selected for fusion are lymphocytes and their normal differentiated progeny, taken either from lymph node tissue or spleen tissue from immunized animals. Applicants prefer to employ immune spleen cells, since they offer a more concentrated and convenient source of antibody producing cells with respect to the mouse system. The myeloma cells provide the basis for continuous propagation of the fused hybrid. Myeloma cells are tumor cells derived from plasma cells.

The cell lines of this invention can be selected and/or maintained in a variety of nutritionally adequate media. Moreover, the hybridoma cell lines can be stored and preserved in a number of ways, including freezing and storage under liquid nitrogen. Frozen cells can be revived and cultured indefinitely with resumed synthesis and secretion of monoclonal antibody. Monoclonal antibodies may be prepared from supernatants of cultured hybridoma cells or from ascites induced by intra-peritoneal inoculation of hybridoma cells into mice. The secreted antibody is recovered by conventional methods such as precipitation, ion exchange chromatography, affinity chromatography, or the like.

IV. Monoclonal Antibodies

While the invention is demonstrated using mouse monoclonal antibodies, the invention is not so limited; in fact, human antibodies may be used and may prove to be preferable. Such antibodies may be obtained by using human hybridomas or hybridomas from mice where the genes or chromosomes in the mice are replaced by human genes or chromosomes, thus producing human monoclonal antibodies. Additionally, mouse monoclonal antibodies can be “chimerized” or “humanized” by recombinant DNA techniques. See, for example, U.S. Pat. Nos. 4,816,567 and 6,331,415, herein incorporated in their entirety by reference. For descriptions of standard recombinant DNA techniques, see Sambrook et al., “Molecular Cloning”, Second Edition, Cold Spring Harbor Laboratory Press (1987) and Ausubel et al. (Eds) “Current Protocols in Molecular Biology”, Green Publishing Associates/Wiley-Interscience, New York (1990), both of which are herein incorporated by reference in their entireity.

Antibody equivalents are prepared by methods known in the art. For example, fragments of antibodies may be prepared enzymatically from full-length antibodies. Preferably, equivalents of antibodies are prepared from DNA encoding such equivalents. DNA encoding fragments of antibodies may be prepared by deleting all but the desired portion of the DNA that encodes the full-length antibody.

The present invention also encompasses antibody variants. Preferably, the light chain variable region sequence of such antibody variants will have at least about 70% amino acid identity, at least about 75% amino acid identity, at least about 80% amino acid identity, at least about 85% amino acid identity, at least about 90% amino acid identity, at least about 95% amino acid identity, at least about 95% amino acid identity, at least about 96% amino acid identity, at least about 97% amino acid identity, at least about 98% amino acid identity, or at least about 99% amino acid identity to the light chain variable region amino acid sequence set forth in SEQ ID NO:1. The heavy chain variable region sequence of the antibody variants will have at least about 70% amino acid identity, at least about 75% amino acid identity, at least about 80% amino acid identity, at least about 85% amino acid identity, at least about 90% amino acid identity, at least about 95% amino acid identity, at least about 95% amino acid identity, at least about 96% amino acid identity, at least about 97% amino acid identity, at least about 98% amino acid identity, or at least about 99% amino acid identity to the heavy chain variable region amino acid sequence set forth in SEQ ID NO:2.

To determine the percent identity of two amino acid sequences, the sequences are aligned for optimal comparison purposes (e.g., gaps can be introduced in one or both of a first and a second amino acid for optimal alignment and non-homologous sequences can be disregarded for comparison purposes). The amino acid residues at corresponding amino acid positions are then compared. When a position in the first sequence is occupied by the same amino acid residue or nucleotide as the corresponding position in the second sequence, then the molecules are identical at that position. The percent identity between the two sequences is a function of the number of identical positions shared by the sequences, taking into account the number of gaps, and the length of each gap, which need to be introduced for optimal alignment of the two sequences.

The comparison of sequences and determination of percent identity between two sequences can be accomplished using a mathematical algorithm. In a preferred embodiment, the percent identity between two amino acid sequences is determined using the Needleman and Wunsch (1970) J. Mol. Biol. 48:444-453 algorithm which has been incorporated into the GAP program in the GCG software package (available at http://www.gcg.com), using the BLOSSUM62 matrix (see Heinkoff et al. (1989) Proc. Natl. Acad. Sci. USA 89:10915), with a gap open penalty of 8 and a gap extension penalty of 2.

Antibody variants of the invention may be created using methods of mutagenesis know in the art, for example, oligonucleotide-mediated (site-directed) mutagenesis, alanine scanning, or DNA shuffling. In one embodiment, an antibody variant comprises at least one light chain variable region sequence that contains substitutions, deletions, or insertions of one or more codons in comparison with the reference amino acid sequence set forth in SEQ ID NO:1. In another embodiment, an antibody variant comprises at least one heavy chain variable region sequence that contains substitutions, deletions, or insertions of one or more codons in comparison with the reference amino acid sequence set forth in SEQ ID NO:1. The claimed antibody variants also comprise embodiments in which both the light chain variable region sequence and the heavy chain variable region sequence of the antibody variant contain substitutions, deletions, or insertions as described. It is preferred that the substitutions, deletions, or insertions do not disrupt the sulfide bridges that link the short chain to the heavy chain, or the first heavy chain to the second heavy chain. It is also preferred that the substitutions, deletions, or insertions do not disrupt the antigen-binding activity (i.e. the PEG-binding activity) of the antibody. Amino acid residues that may be substituted without adversely affecting the antigen-binding activity of the antibody may be determined by systematically substituting each amino acid with another amino acid and testing the resulting variants for antigen-binding activity as described herein.

Amino acid substitutions in the heavy or light chain variable region sequence may be conservative or non-conservative in nature. Preferred antibody variants comprise conservative amino acid substitutions. A “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine).

As described elsewhere herein, the present invention encompasses antibody fragments. Preferred light chain fragments comprise the amino acid sequence set forth in SEQ ID NO:1 and are at least about 110 amino acids in length, at least about 130 amino acids in length, at least about 150 amino acids in length, at least about 170 amino acids in length, at least about 190 amino acids in length, or at least about 210 amino acids in length. Preferred heavy chain fragments comprise the amino acid sequence set forth in SEQ ID NO:2 and are at least about 110 amino acids in length, at least about 130 amino acids in length, at least about 150 amino acids in length, at least about 170 amino acids in length, at least about 190 amino acids in length, at least about 210 amino acids in length, at least about 230 amino acids in length, at least about 250 amino acids in length, at least about 270 amino acids in length, at least about 290 amino acids in length, at least about 310 amino acids in length, at least about 330 amino acids in length, at least about 350 amino acids in length, at least about 370 amino acids in length, at least about 390 amino acids in length, at least about 410 amino acids in length, or at least about 430 amino acids in length. Antibody fragment variants are also encompassed.

V. Polynucleotides

The present invention provides polynucleotides encoding the heavy or light chain variable region amino acid sequences of a monoclonal antibody that binds selectively to PEG as well as variants of these polynucleotides. By “variants” polynucleotides is intended substantially similar sequences. Substantially similar sequences include polynucleotides that, because of the degeneracy of the genetic code, encode the light or heavy chain amino acids sequences of the invention, for example the light chain variable region amino acid sequence shown in SEQ ID NO:1, or the heavy chain variable region amino acid sequence shown in SEQ ID NO:2. Substantially similar sequences encompassed by the present invention also include polynucleotides encoding the antibody variants and antibody fragments described elsewhere herein. Generally, variants of a particular nucleotide sequence of the invention will have at least about 40%, 50%, 60%, 65%, 70%, generally at least about 75%, 80%, 85%, 90%, 91%, 92%, 93%, 94%, 95%, 96%, 97%, 98%, 99% or more sequence identity to the nucleotide sequences set forth in SEQ ID NO:3 or SEQ ID NO:4 as determined by sequence alignment programs described elsewhere herein using default parameters. In a preferred embodiment, the percent identity between two nucleotide sequences is determined using the GAP program in the GCG® Wisconsin Package™. (Accelrys, Burlington Mass.), using a NWSgapdna.CMP matrix and a gap weight of 50 and a length weight of 3.

VI. Modified Antibodies

Modifications of the antibody of the present invention are included within the scope of this invention. One type of covalent modification includes reacting targeted amino acids or carbohydrates with nonpeptidic derivatizing agents including but not limited to those monofunctional, homobifunctional or heterobifunctional reagents described in the Pierce Chemical Company Catalog. Another covalent modification includes the use of chelating agents for labeling the antibody with linkers to attach enzymes, for example horseradish peroxidase or alkaline phosphatase.

Another type of covalent modification of the antibody of the present invention comprises linking the antibody with one of a variety of nonproteinaceous polymers, e.g. polyalkylene glycols, polysaccharides, polyvinylpyrolidone, polyacryomorpholine and the like.

Yet another type of covalent modification of the antibody of the present invention is contemplated which comprises altering the native glycosylation pattern of the polypeptide. Altering the native glycosylation pattern may include deleting one or more carbohydrate moieties found in the native sequence either by removing the underlying glycosylation site or by deleting the glycosylation by chemical and/or enzymatic means. The glycosylation pattern can also be altered by addition of glycosylation sites by altering the amino acid sequence to incorporate another glycosylation site or by chemical and/or enzymatic coupling to the native polypeptide. Such methods are described in the art, e.g. Filpula, D., et al., Protein Engineering, 11(12), 1277-1283, 1998 and Leung, S., et. al., in WO 99/24472.

VII. Methods-for Using Anti-PEG Antibodies

An anti-PEG antibody can be used to isolate molecules containing at least one PEG group by standard techniques, such as affinity chromatography or immunoprecipitation. Moreover, an anti-PEG antibody can be used to detect PEG group-containing molecules. Detection can be facilitated by coupling (i.e., physically linking) the antibody to a detectable substance (i.e., antibody labeling). Examples of detectable substances include various enzymes, prosthetic groups, fluorescent materials, luminescent materials, bioluminescent materials, and radioactive materials. Examples of suitable enzymes include horseradish peroxidase, alkaline phosphatase, β-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin, and examples of suitable radioactive material include ¹²⁵I, ¹³¹I, ³⁵S or ³H.

An anti-PEG antibody or conjugate thereof can be used to detect PEG or PEG molecules in vivo or ex vivo. After a predetermined amount of time after a PEG-modified molecule is administered to a patient, an anti-PEG antibody, having a detectable substance attached such as a DTPA (diethylenetriamine-N,N,N′,N″-tetraacetic acid) complex of ¹³¹I, is administered to a patient and imaged by whole-body scintigraphy or computed tomography. Alternatively, blood or tissue can be taken from the patient and assessed ex vivo.

Yet another diagnostic application for the anti-PEG IgG antibody or functionally equivalent moiety thereof is the use in ELISA. There are many protocol derivations of an ELISA, all of which are included into the present invention. As an example, an anti-PEG antibody is deposited on a surface of a microplate well then washed to remove unbound antibody. A sample containing PEG or a PEG molecule is applied to the well and washed to remove unbound PEG or PEG molecule. A second anti-PEG IgG having a detectable label (ex. horseradish peroxidase) is applied to the microplate well and residual conjugate is removed by washing. In cases where the detectable label is an enzyme, a substrate is added and the resulting product is measured. The measured absorbance is related to the concentration of the PEG or PEG-molecule.

An anti-PEG antibody can be used in therapeutic applications to extend the circulation half-life of PEG-modified molecules. Many antibodies have long half-lives in circulation. In fact, three out of the four subclasses of IgG have half-lives of about 20 days in humans. Each anti-PEG IgG molecule contains two antigen-binding sites per molecule for PEG. A PEG-molecule with only one combining site for the antibody is incapable of crosslinking antibodies and hence would not be subject to normal immune clearance. Therefore, attaching one PEG-molecule to one PEG-binding site on the anti-PEG IgG antibody will result in extended circulation of the PEG-molecule. Examples of using antibodies as carrier proteins are known in the art and is reviewed by Rehlaender and Cho in Pharmaceutical Research, 15(11), 1652-1656, 1998, which is incorporated by reference in its entirety. Preferably human or humanized antibody molecules are used in the invention.

Additionally, an anti-PEG antibody can be used to enhance the clearance of a PEG-modified molecule. A PEG-molecule with multiple combining sites for the antibody, whether a large molecular weight PEG or multiple PEG chains per molecule, is subject to crosslinking and hence would rapidly be cleared from circulation. An example of the utility of having a clearing agent for a molecule in circulation is described in U.S. Pat. No. 5,624,896, which is incorporated by reference in its entirety.

### EXAMPLES

The following examples are offered for purposes of illustration, not by way of limitation.

### Example 1

### Preparation of the Antigen

Bovine serum albumin was used as the carrier protein for the poly(ethylene glycol) hapten. Poly(ethylene glycol) was attached to the ε-amino groups of lysine and the N-terminus by a secondary amine. Use of PEG-tresylate resulted in direct coupling of PEG to the protein without a linker between the PEG chain and the protein's amino groups. Bovine serum albumin (MW=67 kDa, 16 mg, 2.4 E-7 moles) was dissolved in 16 ml of 50 mM sodium phosphate buffer, pH 7.5, containing 150 mM NaCl. For low degree of PEGylation, approximately 3.0 mg (6.0 E-7 moles) of PEG(5,000)-tresylate was added to 4.0 ml BSA solution and mixed at room temperature overnight. For a moderate degree of PEGylation, approximately 6.0 mg (1.2E-6 moles) of PEG(5,000)-tresylate was added to 4.0 ml BSA solution and mixed at room temperature overnight. For high degree of PEGylation, approximately 12.0 mg (2.4E-6 moles) of PEG(5,000)-tresylate was added to 4.0 ml BSA solution and mixed at room temperature overnight.

The purity of the PEG-BSA preparations was determined by gel and capillary electrophoresis. Unmodified PEG reagent was removed by ultrafiltration using an Amicon PM30 membrane with six 50 ml-cycles of 50 mM sodium phosphate, 150 mM NaCl, pH 7.5 buffer. Final concentration was determined by UV absorbance at 280 nm.

### Example 2

### Immunization Protocol

Each of the samples (low, moderate and high) above was used to immunize 5 female Swiss mice. On day, zero roughly 50 μg (0.1 ml) of antigen (PEG-BSA) along with 0.1 ml of Complete Freund's Adjuvant (CFA) was injected intraperitoneally (i.p.) into mice. Three weeks later another 50 μg (0.1 ml) of antigen along with 0.1 ml of Incomplete Freund's Adjuvant (IFA) was administered i.p. into mice. Mice were boosted at six and nine weeks with 50 μg (0.1 ml) of antigen along with 0.1 ml of IFA. Mice were bled at weeks five, eight and eleven.

### Example 3

### Cell Fusion and Hybridoma Expansion Protocol

The mouse selected for fusion was boosted with the same dose of antigen used in previous immunizations. The boost was given four days prior to splenectomy and cell fusion. The antigen preparation was given intraperitoneally without adjuvant.

On the day of fusion the mouse was sacrificed and the spleen removed aseptically. The spleen was minced using forceps and strained through a sieve. The cells were washed twice using IMDM (Iscove's Modified Dulbecco's Medium) medium and counted using a hemocytometer.

The mouse myeloma cell line P3×63Ag8.653 was removed from static, log-phase culture, washed with IMDM and counted using a hemocytometer.

Myeloma and spleen cells were mixed in a 1:5 ratio, centrifuged and the supernatant was discarded. The cell pellet was gently resuspended by tapping the bottom of the tube. One milliliter of a 50% solution of PEG (MW 1450) was added drop by drop over a period of 30 seconds. The pellet was then mixed gently for 30 seconds using a pipette and the resulting cell suspension was allowed to stand undisturbed for another 30 seconds. Five milliliters of IMDM was added over a period of 90 seconds followed by another 5 ml immediately. The resulting cell suspension was left undisturbed for 5 minutes and then spun. The pellet was resuspended in HAT medium (IMDM containing 10% FBS (Fetal Bovine Sera), 2 mM L-glutamine, 0.6% 2-mercaptoethanol (0.04% solution), hypoxanthine, aminopterin, thymidine, and 10% Origen growth factor). The cells were resuspended to 5×10⁵ cells per milliliter and plated on 96-well plates. Two hundred microliters or 1×10⁵ cells were added to each well.

Plates were incubated at 37° C. in a 7% CO₂ atmosphere with 100% humidity. Seven days after fusion, the media was removed and replaced with IMDM containing 10% FBS, 2 mM L-glutamine, 0.6% 2-mercaptoethanol stock (0.04%), hypoxanthine and thymidine. Typically, growing colonies of hybridomas were seen microscopically about seven days after the fusion. These colonies could be seen with the naked eye approximately 10-14 days after fusion.

Ten to fourteen days after fusion, the supernatant was taken from wells with growing hybridoma colonies. The volume of supernatant was approximately 150-200 microliters and contained 10-100 micrograms of antibody per milliliter. This supernatant was tested for PEG-selective antibody using the same assay(s) used to screen the sera. Positive hybridoma colonies were moved from the 96-well plate to a 24-well plate. Three to five days later, the supernatant from 24-well plate was tested to confirm the presence of selective antibody. The volume of supernatant from one well of a 24-well plate was approximately 2 mL and contained 10-100 micrograms/mL of antibody. Cells from positive wells were expanded in T-25 and T-75 flasks and frozen.

Cells from positive wells were cloned by limiting dilution. Hybridoma cells were plated onto 96-well plates at a density of 0.25 cells per well or one cell in every fourth well. Growing colonies were tested 10-14 days later using the same assay(s) used to initially select the hybridomas. Positive clones were expanded and frozen.

### Example 4

### ELISA Analysis of PEG-Selective Antibodies

Hybridoma supernatants were tested for presence of antibodies selective for PEG by ELISA as follows. CovaLink 96-well plates containing surface amino groups were coated with antigen (i.e. various PEG chemistries, molecular weights, and structures (branched versus linear)) along with appropriate controls. Plates were washed several times with deionized water and incubated with 150 μL/well of 1% casein in PBS at RT for 1 hour. The casein solution was discarded and 100 μl of antibody solution (in PBS containing 1% casein and 0.05% Tween 20) was dispensed per well and allowed to stand for 1-2 hours at RT. Each well was washed four times with PBS containing 0.05% Tween 20. Approximately 100 μl of various HRP-anti-mouse antibodies selective for various antibody isotypes in PBS containing 1% casein and 0.05% Tween 20 were added to each well. The appropriate anti-mouse antibody determined the isotype of the PEG selective antibodies. Each well was washed 4 times with PBS containing 0.05% Tween 20 and incubated with 100 μL of substrate (3,3′,5,5′-tetramethylbenzidine for HRP) for 1-10 minutes at RT. Absorbance was read at 650 nm. Results showed that PEG selective antibodies of the present invention have the IgGlκ isotype.

### Example 5

### Selectivity of Monoclonal Antibody 157D 29G1

BSA-PEG (20 kDa) and Lys-PEG (20 kDa) were prepared by reacting a PEG-succinimidyl propionate (SPA; Shearwater Corp., Huntsville, Ala.) derivative with the protein or amino acid in phosphate buffered saline. A mouse anti-PEG monoclonal antibody, clone 157D 29G1, isolated from one of the hybridoma cell lines described above was used in Western Blot analysis.

Each protein sample was prepared at a concentration of 2 mg/ml. For Coomassie blue staining, 3 μl (6 μg) of sample was added to a solution containing 17 μl water and 20 μl sample cocktail buffer (4 ml 10% sodium dodecyl sulfate (SDS), 2 ml glycerol, 250 μl 1M Tris-HCl (pH 6.8), 1 ml β-mercaptoethanol, 1 ml 0.04% bromophenol blue and 1.75 ml water), boiled for 5 min, and electrophoresed in a 10% gel.

For Western Blot analysis, 1 μl (2 μg) of sample was used. After SDS-PAGE and transfer to a nitrocellulose membrane, the blot was incubated in 3% non-fat milk/TNT (0.05M Tris pH 7.4, 0.15M NaCl, 0.01% Tween-20) buffer overnight at 4° C., washed with TNT buffer and incubated in mouse anti-PEG monoclonal antibodies at concentration 1 μg/ml in 1% non-fat milk/TNT for 2 h at room temperature. The membrane was then washed 5 times with TNT buffer, incubated 100 minutes with peroxidase-labeled goat anti-mouse IgG (1:10,000 dilution) in 1% non-fat milk/TNT, washed again and then visualized by chemiluminescent assay.

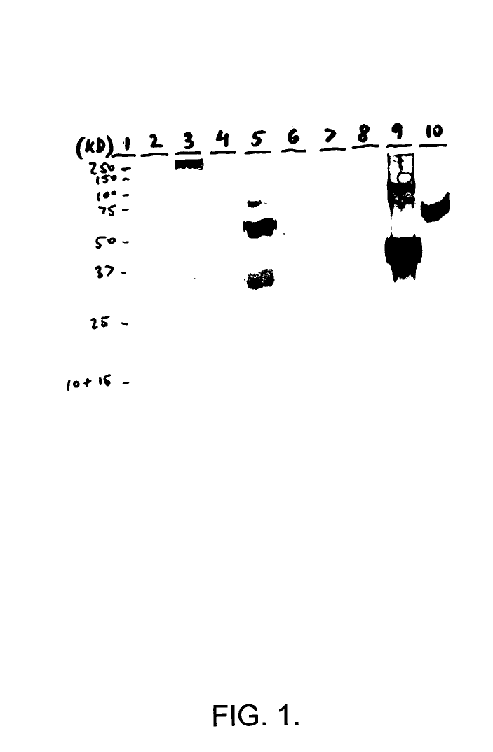

FIG. 1 shows the results of the Western Blot analysis. Samples and molecular weight markers were placed on the gel in the following order: lane 1, prestained protein standard; lane 2, BSA; lane 3, BSA-PEG; lane 4, Lys; lane 5, Lys-PEG; and lanes 6 to 10, PEG with molecular weights of 2, 5, 10, 20 and 30 kDa, respectively. BSA-PEG in lane 3 was detected as single band and Lys-PEG in lane 5 was detected as four bands. The anti-PEG monoclonal antibody also detected PEG in lane 9 and 10 with molecular mass 20 and 30 kDa, respectively. In the Western Blot, these antibodies did not detect PEG with molecular mass 2, 5 or 10 kDa, but 5 kDa PEG covalently bound to either BSA or lysozyme was detected. Since the antibodies were raised against a PEG-protein conjugate that did not contain a linker between the PEG and the protein, the anti-PEG antibody is not binding to the linker between the PEG-BSA and PEG-Lys samples.

As a control, Western Blots were run on cell lysates from hybridoma cell lines producing control (i.e. not anti-PEG) antibodies and no protein was detected with the 157D 29G1 monoclonal antibody.

In an ELISA analysis for 157D 29G1 selectivity, 20 and 30 kDa PEGs were detected with equal titer, 5 and 10 kDa PEGs were detected with lower titer and 2 kDa PEG was not detected using the same antibodies. PEG having a molecular weight of 5 kDa covalently bound to BSA was of equal titer as free 30 kDa PEG.

This hybridoma cell line producing the 157 D29G1 antibody has been deposited with the American Type Culture Collection (ATCC), 10801 University Boulevard, Manassas, Va., on Mar. 1, 2002, and assigned Patent Deposit Number PTA-4112. This deposit will be maintained under the terms of the Budapest Treaty on the International Recognition of the Deposit of Microorganisms for the Purposes of Patent Procedure. This deposit was made merely as a convenience for those of skill in the art and is not an admission that a deposit is required under 35 U.S.C. §112.

### Example 6

### RNA Isolation

RNA was isolated from a hybridoma cell line expressing IgGlκ antibodies selective for PEG, by the method of Chomczynski and Sacchi (1987), Analytical Biochemistry 162, 156-159, with only minor modifications. Briefly, the hybridomas were lysed directly in the culture flask using 2.0 ml of lysis solution (0.36 ml of β-mercaptoethanol plus 50 ml of a stock solution containing 100 g of guanidine thiocyanate, 7.04 ml of 0.75M sodium citrate, pH 7.0, 10.56 ml of 10% sarcosyl and 117.2 ml of sterile water) per 80 cm² of culture dish area. For each ml of lysis solution added in the first step, 0.1 ml of 2M sodium acetate, pH 4.0 and then 1 ml of phenol-chloroform-isoamyl alcohol solution (125:24:1) was added. The solution was vortexed for 10 seconds followed by the addition of the phenol-chloroform mixture and placed on ice for 15 minutes. The solution was centrifuged at 10,000× g for twenty minutes at 4° C. and then the aqueous phase, containing the RNA, was transferred to a new tube. 0.5 ml isopropanol was added for each ml of lysis solution, and the sample was precipitated at −20° C. for 1.5 hours. The precipitated RNA was recovered by centrifugation at 10,000× g for 20 minutes at 4° C. The pellet was dissolved in 0.3 ml lysis solution and then precipitated with isopropanol at −20° C. for one hour. Following centrifugation (10,000× g, 20 minutes, 4° C.) the pellet was resuspended in 75% ethanol (prepared with diethylpyrocarbonate-treated water) and centrifuged as before. The recovered RNA was allowed to air dry for approximately 25 minutes, dissolved in 50 μl of DEPC-treated water, and stored at −80° C.

The RNA was visualized on a denaturing agarose-formaldehyde gel. The concentration of total RNA was determined by measuring the absorbance at 260 nm and found to be approximately 5 μg/μl.

### Example 7

### cDNA Synthesis and Amplification of the Variable Regions of Anti-PEG Antibody

First strand DNA synthesis was accomplished using the 3′primers from the Mouse Ig Prime Kit (Novagen, Madison, Wis.) selective for the murine IgG heavy chain variable region and the murine IgG κ light chain variable region. For a 10 μl reaction, 1 μg of total RNA and 10 pmol of a 3′ primer in 4.75 μl of RNase-free water were denatured at 65° C. for 10 minutes. Then 2 μl of 5× RT Buffer (500 mM Tris-HCl, pH 8.3, 200 mM KCl, 50 mM MgCl₂, 2.5 mM spermidine, Invitrogen, Carlsbad, Calif.), 0.5 μl of dNTP mix (10 mM each, Invitrogen, Carlsbad, Calif.), 0.5 μl of sodium pyrophosphate (80 mM) and 0.25 μl of AMV reverse transcriptase (Invitrogen, Carlsbad, Calif.) were added. The reaction was incubated at 42° C. for one hour followed by inactivation of the reverse transcriptase by heating the reaction at 70° C. for 15 minutes.

The DNA coding for the variable regions of the IgG antibody were amplified via the polymerase chain reaction. For each reaction, 37.75 μl of RNase-free water 5.0 μl of 10× Taq buffer (Eppendorf, Westbury, N.Y.), 1.0 μl of dNTP mix (10 mM each, Invitrogen, Carlsbad, Calif.), 0.25 μl of Taq DNA polymerase (Eppendorf, Westbury, N.Y.) along with 1.0 μl of the appropriate 5′ primers from the Mouse IgG Prime Kit (Novagen, Madison, Wis.) were combined in a 0.2 ml PCR tube. An amplification reaction of one minute at 94° C., 1 minute at 60° C., 2 minutes at 72° C. was repeated 35 times with a final elongation at 72° C. for ten minutes. A 1% agarose gel was used to determine which templates had been successfully amplified. Positive reactions resulted in bands of approximately 450 base pairs in length.

### Example 8

### Cloning of Amplified Variable Chain Fragments

Positive amplification reactions were cloned into a T/A cloning vector, pCR®4-TOPO (Invitrogen, Carlsbad, Calif.) according to the manufacturer's instructions. Each cloning reaction consisted of 1.0 μl of the amplification reaction, 1.0 μl of salt solution (1.2 M NaCl, 0.06 M MgCl₂), 2.0 μl of sterile H₂O and 1.0 μl of the vector. After incubation at room temperature for 15 minutes, the cloning reactions were transformed into TOP10 Escherichia coli (Invitrogen, Carlsbad, Calif.). Two μl of each of the cloning reactions was added to a tube of E. coli. The cells were incubated on ice for 15 minutes. Following a heat shock step (42° C., 30 seconds), the tubes were placed on ice. Then 250 μl of a rich media (SOC) was added to each tube and the reactions were incubated at 37° C. for one hour with shaking at 200 rpm. For each transformation, 50 μl and 100 μl of cells were plate on Luria Broth plates containing 50 μg/ml ampicillin. The plates were placed at 37° C. overnight.

A whole-cell PCR screen using primers flanking the insertion site on the vector identified positive clones. The samples were heated to 94° C. for 10 minutes to lyse the cells. This was followed by an amplification reaction cycle (94° C. for one minute, 55° C. for 1 minute, 72° C. for 1 minute) repeated 30 times, with a final elongation at 72° C. for ten minutes. Colonies that yielded amplification products of about 650 base pairs in length were grown overnight in LB containing 50 μg/ml of ampicillin. Plasmid DNA was recovered according to standard protocols and sent to Research Genetics (Huntsville, Ala.) for sequencing. The sequences of the light and heavy chain variable regions are displayed in SEQ ID NO: 3 and SEQ ID NO: 4.

### Example 9

### Amino Acid Sequencing of Anti-PEG Antibody

The first twenty amino acids of the light and heavy chains of he anti-PEG antibody were sequenced from the N-terminus using 20 cycles of Edman degradation. Results displayed in SEQ ID NO: 1 and SEQ ID NO: 2 show a perfect match to the DNA sequence obtained above.

All publications and patent applications mentioned in the specification are indicative of the level of those skilled in the art to which this invention pertains. All publications and patent applications are herein incorporated by reference to the same extent as if each individual publication or patent application was specifically and individually indicated to be incorporated by reference.

Although the foregoing invention has been described in some detail by way of illustration and example for purposes of clarity of understanding, it will be obvious that certain changes and modifications may be practiced within the scope of the appended embodiments.

That which is claimed:

1. A purified IgG monoclonal antibody that binds selectively to poly(ethylene glycol).

2. A purified IgG monoclonal antibody fragment that binds selectively to poly(ethylene glycol).

3 The antibody fragment of claim 2, wherein the fragment is a F(ab′)₂, Fab, or Fv fragment.

4. The antibody of claim 1, wherein said antibody comprises the V_{L} region amino acid sequence set forth in SEQ ID NO: 1.

5. The antibody of claim 1, wherein said antibody comprises the V_{H} region amino acid sequence set forth in SEQ ID NO: 2.

6. The antibody of claim 1, wherein said antibody comprises at least one light chain having an amino acid sequence having at least about 70% sequence identity to the amino acid sequence set forth in SEQ ID NO:1 and at least one heavy chain having at least about 70% sequence identity to the amino acid sequence set forth in SEQ ID NO:2.

7. The antibody of claim 6, wherein said antibody comprises at least one light chain having an amino acid sequence having at least about 80% sequence identity to SEQ ID NO:1 and at least one heavy chain having at least about 80% sequence identity to SEQ ID NO:2.

8. The antibody of claim 7, wherein said antibody comprises at least one light chain having an amino acid sequence having at least about 90% sequence identity to SEQ ID NO:1 and at least one heavy chain having at least about 90% sequence identity to SEQ ID NO:2.

9. The antibody of claim 8, wherein said antibody comprises at least one light chain having an amino acid sequence having at least about 95% sequence identity to SEQ ID NO:1 and at least one heavy chain having at least about 95% sequence identity to SEQ ID NO:2.

10. The purified IgG monoclonal antibody of claim 1, wherein said antibody is humanized.

11. The antibody of claim 1, wherein said antibody is a human antibody.

12. A molecule comprising the antibody of claim 1 and a detectable label group.

13. A polynucleotide comprising a contiguous series of nucleotides selected from the group consisting of:
a) the contiguous series of nucleotides set forth in SEQ ID NO:3;
b) the contiguous series of nucleotides set forth in SEQ ID NO:4;
c) a contiguous series of nucleotides encoding the light chain variable region amino acid sequence set forth in SEQ ID NO:1;
d) a contiguous series of nucleotides encoding the heavy chain variable region amino acid sequence set forth in SEQ ID NO:2;
e) a polynucleotide encoding a polypeptide having at least about 70% sequence identity with the amino acid sequence set forth in SEQ ID NO:1; and
f) a polynucleotide encoding a polypeptide comprising an amino acid sequence having at least about 70% sequence identity with the amino acid sequence of SEQ ID NO:2.

14. An expression vector comprising a polynucleotide of claim 13.

15. A host cell comprising the expression vector of claim 14.

16. A hybridoma cell line that produces a monoclonal IgG antibody that binds selectively to PEG.

17. The hybridoma cell line of claim 16, wherein said cell line is the cell line deposited with the ATCC as Patent Deposit Number PTA-4112.

18. A purified IgG monoclonal antibody that binds to the same epitope as the monoclonal antibody produced by the hybridoma cell line deposited with ATCC as Patent Deposit Number PTA-4112.

19. The antibody produced by the hybridoma cell line of claim 17.

20. A method of producing the monoclonal IgG antibody of claim 1 comprising culturing a host cell that expresses said antibody.

21. A purified IgG monoclonal antibody produced by the method of claim 20.

22. The purified monoclonal IgG antibody of claim 21, wherein said purified monoclonal antibody is humanized.

23. The purified monoclonal IgG antibody of claim 22, wherein said purified monoclonal antibody is a human antibody.

24. A purified monoclonal IgG antibody produced according to the method of claim 20, wherein said purified IgG monoclonal antibody has a detectable label bound thereto.

25. A composition comprising the monoclonal IgG antibody of claim 1 and a pharmaceutically acceptable carrier.

26. A method for detecting a target comprising at least one poly(ethylene glycol) group, the method comprising:
(a) contacting said target with the purified IgG monoclonal antibody of claim 1 under conditions such that the antibody can bind to the target; and
(b) detecting a complex formed by the binding of said antibody to said target.

27. The method of claim 26, wherein said detecting step comprises performing an immunoassay.

28. The method of claim 27, wherein said antibody is attached to a solid support.

29. A method for purifying a target that comprises at least one poly(ethylene glycol) group, the method comprising the steps of:
(a) contacting the target with the antibody of claim 1 under conditions such that the antibody binds to the target; and
(b) recovering a complex containing the antibody and the bound target.

30. The method of claim 29, wherein the target is selected from the group consisting of peptides, proteins, enzymes, cytokines, hematopoietins, growth factors, hormones, antigens, antibodies, antibody fragments, receptors, protein fragments, drugs, dyes, nucleotides, oligonucleotides, saccharides, polysaccharides, natural polymers, synthetic polymers, lipids, and phospholipids.

31. A method for modulating the number of poly(ethylene glycol) group-containing molecules circulating in a subject, the method comprising the step of administering the purified IgG monoclonal antibody of claim 1 to the subject.

32. The method of claim 31, whereby as a result of said administering the number of poly(ethylene glycol) group-containing molecules is decreased.

33. The method of claim 31, whereby as a result of said administering the number of poly(ethylene glycol) group-containing molecules is increased.


SEQUENCE LISTING (4 sequences)

SEQ ID NO: 1 - protein, 114 aa, Mus musculus
DVLMTQTPLSLPVSLGDQASISCRSSQSIVHSNGNTYLDWYLQKPGQSPKLLIYKVSNRFSGVPDRFSGRGSGTDFTLKITRVEAEDLGVYYCFQGSHVPLTFGAGTKLELKRA

SEQ ID NO: 2 - protein, 119 aa, Mus musculus
QVQLQQPGAELVKPGASVKLSCKASGYIFTNYWINWVKQRPGQGLEWIGNSYPGSSSTNYNEKFKSKATLTVDTSSSTAYMQLSSLTSDDSAVFYCARSPTGTAWFASWGQGTLVTVSA

SEQ ID NO: 3 - DNA, 342 bp, Mus musculus
gatgttttgatgacccaaactccactctccctgcctgtcagtcttggagatcaagcctccatctcttgcagatctagtcagagcattgtacatagtaatggaaacacctatttagattggtacctgcagaaaccaggccagtctccaaagctcctgatctacaaagtttccaaccgattttctggggtcccagacaggttcagtggcaggggatcagggacagatttcacactcaagatcaccagagtggaggctgaggatctgggagtttattactgctttcaaggttcacatgttccgctcacgttcggtgctgggaccaagctggagctgaaacgggct

SEQ ID NO: 4 - DNA, 360 bp, Mus musculus
caggtccaactgcagcagcccggtgctgagcttgtgaagcctggggcctcagtgaagctgtcctgcaaggcttctggctacattttcaccaattattggataaactgggtgaagcagaggcctggacaaggccttgagtggattggaaattcttatcctggtagtagtagtactaactacaatgagaagttcaagagcaaggccacactgactgtagacacatcctccagtacagcctacatgcagctcagcagcctgacatctgacgactctgcggtcttttattgtgcaagatcggggccaactgggacggcctggtttgcttcctggggccaagggactctggtcactgtctctgca